(19) United States
(12) Patent Application Publication
(10) Pub. No.: US 2004/0175737 A1
(43) Pub. Date: Sep. 9, 2004

(54) Assay for RNase H activity

(75) Inventors: Matthew W. Olson, Bardonia, NY (US); John F. O'Connell, Cedar Grove, NJ (US)

Correspondence Address: DARBY & DARBY P.C., P.O. BOX 5257, NEW YORK, NY, 10150-5257, US

(73) Assignee: Wyeth, Madison, NJ

(21) Appl. No.: 10/741,554

(22) Filed: Dec. 19, 2003

Related U.S. Application Data
(60) Provisional application No. 60/436,125, filed on Dec. 23, 2002.

(51) Int. Cl. C12Q 1/68; C12P 19/34

(52) U.S. Cl. 435/6; 435/91.2

(57) ABSTRACT

The present invention provides a method of detecting a nuclease-mediated cleavage of a target nucleic acid through hybridizing a target nucleic acid to a fluorescently labeled oligonucleotide probe complementary to the target nucleic acid and containing a flourophor at one terminus and a quenching group at the other terminus. When the probe is unhybridized to the target nucleic acid, the probe adopts a conformation that places the flourophor and quencher in such proximity that the quencher quenches the flourescent signal of the flourophor and formation of the probe-target hybrid causes sufficient separation of the flourophor and quencher to reduce quenching of the flourescent signal of the flourophor. Once hybrized, the method contacts the probe-target hybrid with an agent having nuclease activity in an amount sufficient to selectively cleave the target nucleic acid and thereby release the intact probe. Detecting the release of the probe is then measured by following a decrease in the flourescent signal of the flourophor as compared to the signal of the probe-target hybrid.

This application claims the benefit of U.S. provisional application No. 60/436,125, filed Dec. 23, 2002, which is incorporated by reference herein in its entirety.

### FIELD OF THE INVENTION

The present invention relates to assays capable of detecting and monitoring RNase H activity in real time. More specifically, the invention relates to assays for monitoring enzymatic degradation of an RNA-DNA duplex by fluorescence quenching.

### BACKGROUND OF THE INVENTION

RNase H. RNase H is a known enzyme that degrades RNA hybridized to a DNA template. For example, the E. coli RNase H1 enzyme is responsible for the removal of RNA primers from the leading and lagging strands during DNA synthesis. RNase is also an important enzyme for the replication of bacterial, viral and human genomes. For example, the HIV reverse transcriptase holoenzyme has an RNase H activity located at the C-terminus of the p66 subunit (Hansen et al., EMBO J. 1998, 7:239-243), and inhibition of that enzyme activity affects at least three unique points within the virus's life cycle (Schatz et al., FEBS Lett. 1989, 257:311-314; Mizrahi et al., Nucl. Acids Res. 1990, 18:5359-5363; Furfine & Reardon, J. Biol. Chem. 1991, 266:406412). Moreover, mutations that affect HIV RNase H activity also abolish viral infectivity (Tisdale et al., J. Gen. Virol. 1991, 72:59-66), emphasizing the potential utility for that enzyme as an antiviral target.

There is considerable interest in assays and methods that are capable of detecting and monitoring RNase H activity, and in identifying compounds that may affect or modulate that enzyme activity. Yet, existing assays for RNase H activity, as well as other methods to establish whether and to what extent nucleic acid cleavage has occurred, are typically time consuming and laborious. Moreover, existing assays are also discontinuous and cannot monitor the RNase reaction in real time. This is particularly disadvantageous in applications where a user wishes to establish precise kinetic information for the enzyme, such as to characterize the effect(s) of a new inhibitory compound.

Fluorescence Resonance Energy Transfer (FRET). Sequence-specific hybridization of labeled oligonucleotide probes has been used as a means for detecting and identifying selected nucleotide sequences, and labeling of such probes with fluorescent labels has provided a relatively sensitive, nonradioactive means for facilitating the detection of probe hybridization. Recent detection methods employ the process of fluorescence energy transfer (FRET) rather than direct detection of fluorescence intensity for detection of probe hybridization. Fluorescence energy transfer occurs between a donor fluorophore and a quencher dye (which may or may not be a fluorophore) when the absorption spectrum of one (the quencher) overlaps the emission spectrum of the other (the donor) and the two dyes are in close proximity. Dyes with these properties are referred to as donor/quencher dye pairs or energy transfer dye pairs.

The excited-state energy of the donor fluorophore is transferred by a resonance dipole-induced dipole interaction to the neighboring quencher. This results in quenching of donor fluorescence. In some cases, if the quencher is also a fluorophore, the intensity of its fluorescence may be enhanced. The efficiency of energy transfer is highly dependent on the distance between the donor and quencher, and equations predicting these relationships have been developed by Förster (Ann. Phys. 1948, 2:55-75). The distance between donor and quencher dyes at which energy transfer efficiency is 50% is referred to as the Förster distance (RO). Other mechanisms of fluorescence quenching are also known including, for example, charge transfer and collisional quenching.

Energy transfer and other mechanisms which rely on the interaction of two dyes in close proximity to produce quenching are an attractive means for detecting or identifying nucleotide sequences, as such assays may be conducted in homogeneous formats. Homogeneous assay formats are simpler than conventional probe hybridization assays which rely on detection of the fluorescence of a single fluorophor label, as heterogeneous assays generally require additional steps to separate hybridized label from free label. Traditionally, FRET and related methods have relied upon monitoring a change in the fluorescence properties of one or both dye labels when they are brought together by the hybridization of two complementary oligonucleotides. In this format, the change in fluorescence properties may be measured as a change in the amount of energy transfer or as a change in the amount of fluorescence quenching, typically indicated as an increase in the fluorescence intensity of one of the dyes. In this way, the nucleotide sequence of interest may be detected without separation of unhybridized and hybridized oligonucleotides. The hybridization may occur between two separate complementary oligonucleotides, one of which is labeled with the donor fluorophore and one of which is labeled with the quencher. In double-stranded form there is decreased donor fluorescence (increased quenching) and/or increased energy transfer as compared to the single-stranded oligonucleotides.

Several formats for FRET hybridization assays are reviewed in Nonisotopic DNA Probe Techniques (1992, Academic Press, Inc.; See, in particular, pages. 311-352). Alternatively, the donor and quencher may be linked to a single oligonucleotide such that there is a detectable difference in the fluorescence properties of one or both when the oligonucleotide is unhybridized vs. when it is hybridized to its complementary sequence. In this format, donor fluorescence is typically increased and energy transfer/quenching are decreased when the oligonucleotide is hybridized. For example, a self-complementary oligonucleotide labeled at each end may form a hairpin which brings the two fluorophores (i.e., the 5′ and 3′ ends) into close spatial proximity where energy transfer and quenching can occur. Hybridization of the self-complementary oligonucleotide to its complementary sequence in a second oligonucleotide disrupts the hairpin and increases the distance between the two dyes, thus reducing quenching. A disadvantage of the hairpin structure is that it is very stable and conversion to the unquenched, hybridized form is often slow and only moderately favored, resulting in generally poor performance. Tyagi & Kramer (Nature Biotech. 1996, 14:303-308) describe a hairpin labeled as described above which comprises a detector sequence in the loop between the self-complementary arms of the hairpin which form the stem. The base-paired stem must melt in order for the detector sequence to hybridize to the target and cause a reduction in quenching. A “double hairpin” probe and methods of using it are described by Bagwell et al. (Nucl. Acids Res. 1994, 22:2424-2425; See also, U.S. Pat. No. 5,607,834). These structures contain the target binding sequence within the hairpin and therefore involve competitive hybridization between the target and the self-complementary sequences of the hairpin. Bagwell solves the problem of unfavorable hybridization kinetics by destabilizing the hairpin with mismatches.

Homogeneous methods employing energy transfer or other mechanisms of fluorescence quenching for detection of nucleic acid amplification have also been described. (Lee et al., Nuc. Acids Res. 1993, 21:3761-3766) disclose a real-time detection method in which a doubly-labeled detector probe is cleaved in a target amplification-specific manner during PCR. The detector probe is hybridized downstream of the amplification primer so that the 5′-3′ exonuclease activity of Taq polymerase digests the detector probe, separating two fluorescent dyes which form an energy transfer pair. Fluorescence intensity increases as the probe is cleaved.

Signal primers (sometimes also referred to as detector probes) which hybridize to the target sequence downstream of the hybridization site of the amplification primers have been described for homogeneous detection of nucleic acid amplification (U.S. Pat. No. 5,547,861). The signal primer is extended by the polymerase in a manner similar to extension of the amplification primers. Extension of the amplification primer displaces the extension product of the signal primer in a target amplification-dependent manner, producing a double-stranded secondary amplification product which may be detected as an indication of target amplification. Examples of homogeneous detection methods for use with single-stranded signal primers are described in U.S. Pat. No. 5,550,025 (incorporation of lipophilic dyes and restriction sites) and U.S. Pat. No. 5,593,867 (fluorescence polarization detection). More recently signal primers have been adapted for detection of nucleic acid targets using FRET methods. U.S. Pat. No. 5,691,145 discloses G-quartet structures containing donor/quencher dye pairs appended 5′ to the target binding sequence of a single-stranded signal primer. Synthesis of the complementary strand during target amplification unfolds the G-quartet, increasing the distance between the donor and quencher dye and resulting in a detectable increase in donor fluorescence. Partially single-stranded, partially double-stranded signal primers labeled with donor/quencher dye pairs have also recently been described. For example, EP 0 878 554 discloses signal primers with donor/quencher dye pairs flanking a single-stranded restriction endonuclease recognition site. In the presence of the target, the restriction site becomes double-stranded and cleavable by the restriction endonuclease. Cleavage separates the dye pair and decreases donor quenching. EP 0 881 302 describes signal primers with an intramolecularly base-paired structure appended thereto. The donor dye of a donor/quencher dye pair linked to the intramolecularly base-paired structure is quenched when the structure is folded, but in the presence of a target a sequence complementary to the intramolecularly base-paired structure is synthesized. This unfolds the intramolecularly base-paired structure and separates the donor and quencher dyes, resulting in a decrease in donor quenching. Nazarenko, et al. (U.S. Pat. No. 5,866,336) describe a similar method wherein amplification primers are configured with hairpin structures which carry donor/quencher dye pairs.

There exists, therefore, a continuing need for assays and methods that are capable of detecting and/or monitoring degradation of RNA and other nucleic acids, e.g., by enzymes such as RNase H. In particular, there is need for assays and methods that are capable of detecting and monitoring such activity in real time.

The citation of any reference in this section or throughout the text of this application does not constitute an admission that such reference is available as “prior art” to the invention described and claimed herein.

### SUMMARY OF THE INVENTION

The present invention overcomes disadvantages of the prior art by providing a method of detecting a nuclease-mediated cleavage of a target nucleic acid through (a) hybridizing a target nucleic acid to a fluorescently labeled oligonucleotide probe complementary to the target nucleic acid and containing a flourophor at one terminus and a quenching group at the other terminus, wherein (i) when the probe is unhybridized to the target nucleic acid, the probe adopts a conformation that places the flourophor and quencher in such proximity that the quencher quenches the flourescent signal of the flourophor and (ii) formation of the probe-target hybrid causes sufficient separation of the flourophor and quencher to reduce quenching of the flourescent signal of the flourophor; (b) contacting the probe-target hybrid with an agent having nuclease activity in an amount sufficient to selectively cleave the target nucleic acid and thereby release the intact probe; and (c) detecting the release of the probe by measuring a decrease in the flourescent signal of the flourophor as compared to the signal of the probe-target hybrid.

Another embodiment of the invention provides a method for measuring RNase H activity of an agent, by hybridizing a target RNA to a fluorescently labeled oligodesoxyribonucleotide probe complementary to the target RNA and containing a flourophor at one terminus and a quenching at the other terminus, wherein (i) when the probe is unhybridized to the target RNA, the probe adopts a conformation that places the flourophor and quencher in such proximity that the quencher quenches the flourescent signal of the flourophor and (ii) formation of the probe-target hybrid causes sufficient separation of the flourophor and quencher to reduce quenching of the flourescent signal of the flourophor; contacting the probe-target hybrid with the agent in an amount sufficient to selectively cleave the target RNA and thereby release the intact probe; and measuring a decrease in the flourescent signal of the flourophor as compared to the signal of the probe-target hybrid.

In one embodiment, the agent is selected from the group consisting of RNase H, reverse transcriptase, E. coli Rnase H1 and H2, Human RNase H1 and H2, hammerhead ribozymes, HBV reverse transcriptase, and integrase. In a preferred embodiment, the reverse transcriptase is HIV reverse transcriptase. In yet another embodiment, the reverse transcriptase contains a RNase domain.

In an embodiment of the present invention, the probe is DNA, and the target is the DNA:RNA hybrid substrate. Also in an embodiment of the present invention, the probe is at least 18 nucleotides in length.

In the present invention, the probe, when unhybridized to the target nucleic acid or RNA, adopts a hairpin secondary structure conformation that brings the fluorophor and quencher into proximity. In addition, where the RNase H-mediated or nuclease reaction is performed in the presence of a compound, wherein a difference in the rate of the decrease in the flourescent signal of the flourophor during the nuclease reaction, as compared to the decrease observed when the same reaction is conducted in the absence of the compound, the method is indicative of the ability of the compound to either inhibit or enhance the nuclease activity of the agent.

In one embodiment of the invention, the method monitors the flourescent signal of the flourophor during the RNase H-mediated or nuclease reaction.

The present invention also provides a method of screening for a modulator of the nuclease activity of an agent by hybridizing a target nucleic acid to a fluorescently labeled oligonucleotide probe complementary to the target nucleic acid and containing a flourophor at one terminus and a quenching group at the other terminus, wherein (i) when the probe is unhybridized to the target nucleic acid, the probe adopts a conformation that places the flourophor and quencher in such proximity that the quencher quenches the flourescent signal of the flourophor and (ii) formation of the probe-target hybrid causes sufficient separation of the flourophor and quencher to reduce quenching of the flourescent signal of the flourophor; preparing two samples containing the probe-target hybrid; contacting the probe-target hybrid of a first sample with the agent in an amount sufficient to selectively cleave the target nucleic acid and thereby release the intact probe; contacting the probe-target hybrid of a second sample with the agent in an amount sufficient to selectively cleave the target nucleic acid and thereby release the intact probe in the presence of a candidate compound, which is being tested for its ability to modulate the nuclease activity of the agent; detecting the release of the probe in each sample by measuring a decrease in the flourescent signal of the flourophor as compared to the signal of the probe-target hybrid; and comparing the rate of the decrease in the flourescent signal of the flourophor in the two samples, wherein a difference in the rate of the decrease in the flourescent signal of the flourophor during the nuclease reaction in the two samples is indicative of the ability of the compound to either inhibit or enhance the nuclease activity of the agent.

In a preferred embodiment, the a greater extent or relative rate of decrease of the flourescent signal of the flourophor in the second sample compared to the first sample indicates that the candidate compound is an agent agonist. In another embodiment, a lesser extent or relative rate of decrease of the flourescent signal of the flourophor in the second sample compared to the first sample indicates that the candidate compound is an agent antagonist.

The present invention also provides for a kit for measuring a nuclease activity of an agent, comprising a target nucleic acid and a fluorescently labeled oligonucleotide probe complementary to the target nucleic acid and containing a flourophor at one terminus and a quencher at the other terminus, wherein (i) when the probe is unhybridized to the target nucleic acid, the probe adopts a conformation that places the flourophor and quencher in such proximity that the quencher quenches the flourescent signal of the flourophor and (ii) formation of the probe-target hybrid causes sufficient separation of the flourophor and quencher to reduce quenching of the flourescent signal of the flourophor.

In one embodiment of the kit, the probe is at least 18 nucleotides in length. In another embodiment of the kit, the probe, when unhybridized to the target nucleic acid, adopts a hairpin secondary structure conformation that brings the fluorophor and quencher into proximity.

In a preferred embodiment of the kit, the probe is DNA, and the target nucleic acid is DNA:RNA hybrid substrate.

In one embodiment of the kit, the invention also has an agent. In a preferred embodiment, the agent is selected from the group consisting of RNase H, reverse transcriptase, E. coli RNase H1 and H2, Human RNase H1 and H2, hammerhead ribozymes, HBV reverse transcriptase, and integrase. In yet another embodiment, the reverse transcriptase is HIV reverse transcriptase.

The present invention also provides for an assay mixture for measuring a nuclease activity of an agent, comprising a target nucleic acid and a fluorescently labeled oligonucleotide probe complementary to the target nucleic acid and containing a flourophor at one terminus and a quenching group at the other terminus, wherein (i) when the probe is unhybridized to the target nucleic acid, the probe adopts a conformation that places the flourophor and quencher in such proximity that the quencher quenches the flourescent signal of the flourophor and (ii) formation of the probe-target hybrid causes sufficient separation of the flourophor and quencher to reduce quenching of the flourescent signal of the flourophor.

In a preferred embodiment of the assay, the probe is DNA, and the target nucleic acid is RNA. In yet another embodiment, the probe and the target nucleic acid are hybridized to each other to form a probe-target hybrid.

In one embodiment of the assay mixture, there is also an agent. In a preferred embodiment, the agent is selected from the group consisting of RNase H, reverse transcriptase, E. coli RNase H1 and H2, Human RNase H1 and H2, hammerhead ribozymes, HBV reverse transcriptase, and integrase. In a further embodiment, the reverse transcriptase is HIV reverse transcriptase.

### BRIEF DESCRIPTION OF THE FIGURES

FIGS. 1A-1B show PAGE analysis of substrate RNA synthesized by a T7 RNA polymerase reaction. FIG. 1A shows RNA product evaluated on a denaturing (7M Urea-15% polyacrylamide) gel, whereas FIG. 1B shows a non-denaturing (native 15% polyacrylamide) gel. Nucleic acids in both gels were detecting by ethidium bromide staining. The gels in both figures were loaded as follows:

lane 1: 49-mer template DNA (SEQ ID NO:2);

lane 2: control RNA 125-mer;

lane 3-6: RNA derived from the T7 RNA polymerase reaction; and

lane 7: 49-mer template DNA.

FIGS. 2A-2D show radiolabeled RNA-DNA substrate evaluated by PAGE. FIG. 2A illustrates the substrate DNA nucleotide sequence (SEQ ID NO:2) annealed to the substrate RNA (SEQ ID NO: 1). FIG. 2B shows the image of a non-denaturing gel loaded with the unlabeled RNA annealed to ³³P-end labeled DNA. FIGS. 2C and 2D show denaturing and non-denaturing polyacrylamide gels, respectively, that have both been loaded with internally radiolabeled RNA and unlabeled DNA. The method of nucleic acid detection is by phosphoimagery.

FIGS. 3A-3B show results from a PAGE-based assay for RNase H activity. FIG. 3A shows results from an embodiment of the assay in which an unlabeled RNA/end-labeled DNA substrate was used, whereas FIG. 3B shows results for an alternative embodiment that used a labeled RNA/unlabeled DNA substrate.

FIG. 4 shows the image of a polyacrylamide gel loaded with unlabeled RNA/end-labeled DNA hybrid digested in an assay for HIV RT RNase H activity.

FIGS. 5A-5B show plots of HIV RT RNase H activity ascertained from quantitative analysis of the PAGE gels illustrated in FIG. 3A and FIG. 4, respectively.

FIGS. 6A-6C show PAGE gels run with ssRNA substrate that was incubated with (FIG. 6A) or without (FIG. 6B) 1 U (19 fmol≈2.2 ng) HIV RT RNase H enzyme, and a PAGE gel in which 2.5 pmol RNA-DNA hybrid substrate was incubated with the enzyme to verify RNase H activity (FIG. 6C).

FIG. 7 is the PAGE gel from an RNase H assay that was run with polyA (lanes 2-3), polyU (lanes 4-5) and 18S RNA (SEQ ID NO:5; lanes 6-7) along with radiolabeled RNA-DNA hybrid substrate.

FIG. 8 shows the PAGE gel from an RNase H assay that was run with “contaminating oligonucleotides referred to here as Oligo 1 (SEQ ID NO:6; lanes 3-5), Oligo 2 (SEQ ID NO:7, lanes 6-8) and Oligo 3 (SEQ ID NO:8; lanes 9-11).

FIGS. 9A-9D show results from a PAGE-based RNase H assay using HIV RNase H (FIG. 9A), MMLV RNAse H (FIG. 9B) and mutant MMLV RNase H (FIG. 9C). A quantitative analysis of these data is plotted in FIG. 9D.

FIGS. 10A-10C provide a schematic illustration of a preferred, real time RNase H assay of the invention. FIG. 10A illustrates an exemplary RNA substrate (SEQ ID NO: 10) annealed to an exemplary DNA probe (SEQ ID NO:9) that is labeled with a fluorophor moiety (F) and a quencher moiety (Q). The 5′- and 3′ regions of the DNA probe are capable of annealing to each other after the RNA substrate has been digested by RNase H, placing the fluorophor moiety and the quencher moiety in sufficient proximity so that the quencher moiety absorbs at least part of the detectable signal emitted by the fluorophor moiety (FIG. 10B). FIG. 10C illustrates a typical fluorescent signal that may be observed in real time as RNase H degrades the RNA substrate in this assay.

FIGS. 11A-11B are plots of fluorescence intensity measurements from real time RNase H assays of the invention that used HIV RT RNase H (FIG. 11A) and E. coli RNase H1 (FIG. 11B).

### DETAILED DESCRIPTION

The present invention is directed to a method of a fluorometric assay for real-time monitoring of RNase H activity. Specifically, the invention relates to the quantitative assessment of RNase H activity through a decrease in fluorescence.

### Definitions

In accordance with the invention, there may be employed conventional molecular biology, microbiology and recombinant DNA techniques within the skill of the art. Such techniques are explained fully in the literature and the terms used here to describe such techniques will generally have the meaning normally used in the art. See, for example, Sambrook, Fitsch & Maniatis, Molecular Cloning: A Laboratory Manual, Second Edition (1989) Cold Spring Harbor Laboratory Press, Cold Spring Harbor, N.Y. (referred to herein as “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al. (eds.), Current Protocols in Molecular Biology, John Wiley & Sons, Inc. (1994).

The term “fluorescent label” or “fluorophore” as used herein refers to a substance or portion thereof that is capable of exhibiting fluorescence in the detectable range. Examples of fluorophores that can be used according to the invention include fluorescein isothiocyanate, fluorescein amine, eosin, rhodamine, dansyl, umbelliferone, texas red, Cy5, Cy3 and europium. Other fluorescent labels will be known to the skilled artisan. Some general guidance for designing sensitive fluorescent labelled polynucleotide probes can be found in Heller and Jablonski's U.S. Pat. No. 4,996,143. This patent discusses the parameters that should be considered when designing fluorescent probes, such as the spacing of the fluorescent moieties (i.e., when a pair of fluorescent labels is utilized in the present method), and the length of the linker arms connecting the fluorescent moieties to the base units of the oligonucleotide. The term “linker arm” as used herein is defined as the distance in Angstroms from the purine or pyrimidine base to which the inner end is connected to the fluorophore at its outer end.

The term “cleavage that is enzyme-mediated” refers to cleavage of DNA or RNA that is catalyzed by such enzymes as DNases, RNases, helicases, exonucleases, restriction endonucleases, or retroviral integrases. Other enzymes that effect nucleic acid cleavage will be known to the skilled artisan and can be employed in the practice of the present invention. A general review of these enzymes can be found in Chapter 5 of Sambrook et al, supra.

As used herein, the terms “nucleic acid”, “polynucleotide” and “oligonucleotide” refer to primers, probes, oligomer fragments to be detected, oligomer controls and unlabeled blocking oligomers and shall be generic to polydeoxyribonucleotides (containing 2-deoxy-D-ribose), to polyribonucleotides (containing D-ribose) as well as chimeric polynucleotides (containing 2-deoxy-D-ribose and D-ribose nucleotides), and to any other type of polynucleotide which is an N glycoside of a purine or pyrimidine base, or modified purine or pyrimidine bases. There is no conceived distinction in length between the term “nucleic acid”, “polynucleotide” and “oligonucleotide”, and these terms are used interchangeably. Thus, these terms include double-and single stranded DNA, as well as double- and single stranded RNA. Preferably, the oligonucleotides used in connection with assays of this invention will be at least 10 nucleotides in length, and more preferably between about 10 and 100 nucleotides in length, with oligonucleotides between about 25 and 50 nucleotides in length being even more preferred.

The oligonucleotide is not necessarily limited to a physically derived species isolated from any existing or natural sequence but may be generated in any manner, including chemical synthesis, DNA replication, reverse transcription or a combination thereof. The terms “oligonucleotide” or “nucleic acid” refers to a polynucleotide of genomic DNA or RNA, cDNA, semisynthetic, or synthetic origin which, by virtue of its derivation or manipulation: (1) is not affiliated with all or a portion of the polynucleotide with which it is associated in nature; and/or (2) is connected to a polynucleotide other than that to which it is connected in nature; and (3) is unnatural (not found in nature). Oligonucleotides are composed of reacted mononucleotides to make oligonucleotides in a manner such that the 5′ phosphate of one mononucleotide pentose ring is attached to the 3′ oxygen of its neighbor in one direction via a phosphodiester linkage, and is referred to as the “5′end” end of an oligonucleotide if its 5′ phosphate is not linked to the 3′ oxygen of a mononucleotide pentose ring and subsequently referred to as the “3′end” if its 3′ oxygen is not linked to a 5′ phosphate of a subsequent mononucleotide pentose ring. A nucleic acid sequence, even if internalized to a larger oligonucleotide, also may be said to have 5′ and 3′ ends. Two distinct, non-overlapping oligonucleotides annealed to two different regions of the same linear complementary nucleic acid sequence, so the 3′ end of one oligonucleotide points toward the 5′ end of the other, will be termed the “upstream” oligonucleotide and the latter the “downstream” oligonucleotide. In general, “downstream” refers to a position located in the 3′ direction on a single stranded oligonucleotide, or in a double stranded oligonucleotide, refers to a position located in the 3′ direction of the reference nucleotide strand.

The term “primer” may refer to more than one oligonucleotide, whether isolated naturally, as in a purified restriction digest, or produced synthetically. The primer must be capable of acting as a point of initiation of synthesis along a complementary strand (DNA or RNA) when placed under reaction conditions in which the primer extension product synthesized is complementary to the nucleic acid strand. These reaction conditions include the presence of the four different deoxyribonucleotide triphosphates and a polymerization-inducing agent such as DNA polymerase or reverse transcriptase. The reaction conditions incorporate the use of a compatible buffer (including components which are cofactors, or which affect pH, ionic strength, etc.), at an optimal temperature. The primer is preferably single-stranded for maximum efficiency in the amplification reaction.

A complementary nucleic acid sequence refers to an oligonucleotide which, when aligned with the nucleic acid sequence such that the 5′ end of one sequence is paired with the 3′ end of the other. This association is termed as “antiparallel.” Modified base analogues not commonly found in natural nucleic acids may be incorporated (enzymatically or synthetically) in the nucleic acids including but not limited to primers, probes or extension products of the present invention and may include, for example, inosine and 7-deazaguanine. Complementarity of two nucleic acid strands may not be perfect; some stable duplexes may contain mismatched base pairs or unmatched bases and one skilled in the art of nucleic acid technology can determine their stability hypothetically by considering a number of variables including, the length of the oligonucleotide, the concentration of cytosine and guanine bases in the oligonucleotide, ionic strength, pH and the number, frequency and location of the mismatched base pairs. The stability of a nucleic acid duplex is measured by the melting or dissociation temperature, or “Tₘ.” The Tₘ of a particular nucleic acid duplex under specified reaction conditions. It is the temperature at which half of the base pairs have disassociated.

As used herein, the term “target sequence” or “target nucleic acid sequence” refers to a region of the oligonucleotide which is to be either amplified, detected or both. The target sequence resides between the two primer sequences used for amplification or as a reverse transcribed single-stranded cDNA product. The target sequence may be either naturally derived from a sample or specimen or synthetically produced.

As used herein, a “probe” comprises a ribo-oligonucleotide which forms a duplex structure with a sequence in the target nucleic acid, due to complementarity of at least one sequence of the ribo-oligonucleotide to a sequence in the target region. The probe, preferably, does not contain a sequence complementary to the sequence(s) used to prime the polymerase chain reaction (PCR) or the reverse transcription (RT) reaction. The probe may be chimeric, that is, composed in part of DNA. Where chimeric probes are used, the 3′ end of the probe is generally blocked if this end is composed of a DNA portion to prevent incorporation of the probe into primer extension product. The addition of chemical moieties such as biotin, fluorescein, rhodamine and even a phosphate group on the 3′ hydroxyl of the last deoxyribonucleotide base can serve as 3′ end blocking groups and under specific defined cases may simultaneously serve as detectable labels or as quenchers. Furthermore, the probe may incorporate modified bases or modified linkages to permit greater control of hybridization, polymerization or hydrolyzation.

The term “label” refers to any atom or molecule which can be used to provide a detectable (preferably quantifiable) real time signal. The detectable label can be attached to a nucleic acid probe or protein. Labels provide signals detectable by either fluorescence, phosphorescence, chemiluminescence, radioactivity, colorimetric (ELISA), X-ray diffraction or absorption, magnetism, enzymatic activity, or a combination of these.

The term “absorber/emitter moiety” refers to a compound that is capable of absorbing light energy of one wavelength while simultaneously emitting light energy of another wavelength. This includes phosphorescent and fluorescent moieties. The requirements for choosing absorber/emitter pairs are: (1) they should be easily functionalized and coupled to the probe; (2) the absorber/emitter pairs should in no way impede the hybridization of the functionalized probe to its complementary nucleic acid target sequence; (3) the final emission (fluorescence) should be maximally sufficient and last long enough to be detected and measured by one skilled in the art; and (4) the use of compatible quenchers should allow sufficient nullification of any further emissions.

As used in this application, “real time” refers to detection of the kinetic production of signal, comprising taking a plurality of readings in order to characterize the signal over a period of time. For example, a real time measurement can comprise the determination of the rate of increase of detectable product. Alternatively, a real time measurement may comprise the determination of time required before the target sequence has been amplified to a detectable level.

The term “chemiluminescent and bioluminescent” include moieties which participate in light emitting reactions. Chemiluminescent moieties (catalyst) include peroxidase, bacterial luciferase, firefly luciferase, functionlized iron-porphyrin derivatives and others.

As defined herein, “nuclease activity” refers to that activity of a template-specific ribo-nucleic acid nuclease, RNase H. As used herein, the term “RNase H” refers to an enzyme which specifically degrades the RNA portion of DNA/RNA hybrids. The enzyme does not cleave single or double-stranded DNA or RNA and a thermostable hybrid is available which remains active at the temperatures typically encountered during PCR. Generally, the enzyme will initiate nuclease activity whereby ribo-nucleotides are removed or the ribo-oligonucleotide is cleaved in the RNA-DNA duplex formed when the probe anneals to the target DNA sequence.

The term “hybridization or reaction conditions” refers to assay buffer conditions which allow selective hybridization of the labeled probe to its complementary target nucleic acid sequence. These conditions are such that specific hybridization of the probe to the target nucleic acid sequence is optimized while simultaneously allowing for but not limited to cleavage of the probe-target hybrid by a nuclease enzyme or by another agent having a nuclease activity. The reaction conditions are optimized for co-factors, ionic strength, pH and temperature.

### RNase H Molecular Beacon Assay

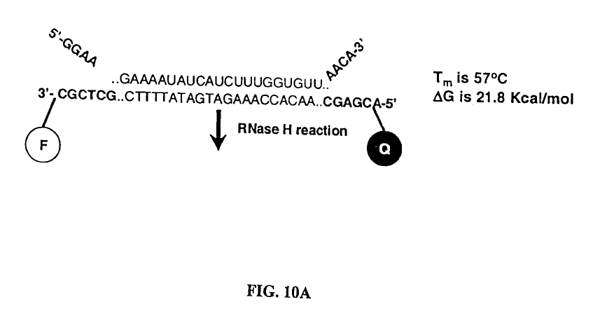

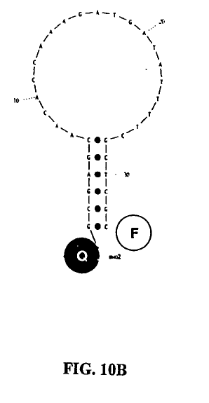

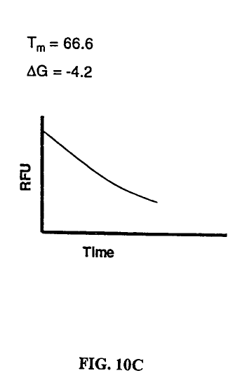

In preferred embodiments, the assays and methods of the present invention detect RNase H activity and/or other nuclease-mediated cleavage of nucleic acids in an assay that is referred to here as a “molecular beacon” assay. An exemplary embodiment of such an assay is illustrated schematically in FIGS. 10A-10C. The assay detects degradation of a nucleic acid substrate which, preferably, is an RNA substrate that is annealed to at least one region or part of an oligonucleotide probe. In preferred embodiments, the oligonucleotide probe is a DNA probe (e.g., a deoxyoligonucleotide probe), which may also be referred to in the context of this invention as the DNA “substrate” moiety. Typically, both the oligonucleotide probe and the RNA substrate will be oligonucleotide molecules that are between about 10 and about 100 nucleotides in length and may be, e.g., between about 10-50 nucleotides in length, more preferably between 15-25 nucleotides length. In preferred embodiments, the oligonucleotide probe is at least 18 nucleotides in length.

FIG. 10A shows an exemplary RNA substrate having the nucleotide sequence set forth in SEQ ID NO: 10 and annealed to an exemplary DNA probe having the nucleotide sequence set forth in SEQ ID NO:9. However, these sequences are only exemplary, for the purposes of illustrating and better explaining the present invention. The actual sequence of the RNA substrate and/or the oligonucleotide probe is not critical and those skilled in the art will be able to readily design other appropriate sequences without undue experimentation.

Nevertheless, the substrate and probe sequences will preferably have certain properties. In particular, the oligonucleotide probe preferably comprises regions of sequences that are referred to here as the 5′-region and the 3′-region and are located at the 5′ and 3′-ends of the oligonucleotide, respectively. These 5′- and 3′-regions preferably comprise nucleotide sequences that are complementary to each other such that, when the oligonucleotide probe is not annealed to a RNA substrate, the two regions may hybridze to each other and thereby form a hairpin loop, such as the exemplary hairpin loop illustrated in FIG. 10B.

The oligonucleotide probe also preferably comprises a third sequence region, which is preferably situated between the probe's 5′-region and its 3′-region, and is therefore referred to here as the “center region” of the oligonucleotide probe. The actual sequence of this center region also is not critical to practicing the present invention. It is sufficient that the center region of the oligonucleotide probe be sufficiently complementary to at least a part of the RNA substrate so that the two molecules are capable of hybridizing to each other under assay conditions.

The oligonucleotide probe used in a molecular beacon assay of this invention may also comprise a detectable label which, in preferred embodiments, comprises a fluorescent or “fluorophor” moiety that emits a detectable fluorescent signal. More preferably, the oligonucleotide probe further comprises a “quencher” quencher moiety which, when positioned in sufficient proximity to the fluorophor moiety, is capable of absorbing at least a part of the fluorescent signal emitted by that fluorophor moiety. Suitable fluorescent labels and appropriate quencher for use therewith are well known in the art. For example, in one preferred embodiment the fluorophor moiety may be fluorescein and the quencher moiety may be dabcyl. Both of these labels are commercially available, e.g., from Stratagene (La Jolla, Calif.). However, a variety of other such moieties are generally available and/or otherwise known in the art, and the use of such other fluorophor and quencher moities is also contemplated in the present invention. Those skilled in the art will be able to readily identify other labels and quenchers that are suitable for and may be used in a molecular beacon or other assay of this invention.

The fluorophor and quencher moities are preferably attached at opposite ends of the oligonucleotide probe. Thus, the exemplary oligonucleotide probe in FIG. 10A is illustrated as having the fluorophor moiety attached to the 3′-region (e.g., on the 3′-end) of the oligonucleotide probe while the quencher moiety is attached to the 5′-region (e.g., on the 5′-end) of the oligonucleotide probe. However, embodiments in which the quencher moiety is attached to the 3′-region and the fluorophor moiety is attached to the 5′-region are also contemplated and generally will be equally preferred.

It therefore is not critical which particular fluorophor or quencher moiety is attached to which particular end of the oligonucleotide probe. However, the two moieties are preferably positioned such that, when the oligonucleotide probe is annealed to the RNA substrate, the fluorophor and quencher moities are sufficiently extraneous from each other that the quencher moiety does not absorb a detectable amount of signal from the fluorophor moiety. However, when the 5′- and 3′-regions of the oligonucleotide probe are hybridized to each other and/or the oligonucleotide probe forms a hairpin loop (as shown, e.g., in FIG. 10B), the fluorophor and quencher moieties should be sufficiently close together so that at least part of the fluorescent signal emitted by the fluorophor is absorbed by the quencher such that the intensity of fluorescent signal from the sample is detectably reduced.

In preferred embodiments therefore, a molecular beacon of the assay will begin with a sample containing an oligonucleotide probe and a RNA substrate under conditions so that the oligonucleotide probe and RNA substrate are annealed to each other, as illustrated in FIG. 10A. An enzyme or other molecule having or suspected of degrading RNA (for example, an RNase H enzyme) may then be added to the sample and, optionally, a test compound suspected of modulating the enzymatic activity may also be added. The probe and substrate are then incubated in the presence of the enzyme and optional test compound, and the fluorescent signal intensity of the sample is measured. Without being limited to any particular theory or mechanism of action, it is understood that, as RNA substrate is digested in the sample, an increasing fraction of the oligonucleotide probes will self-hybridize, e.g., to form hairpin loops as illustrated in FIG. 10B. Thus, as the RNase reaction progresses, an increasing number of the oligonucleotide probes will adopt a conformation where the quencher moiety is brought into close proximity with the fluorophor moiety, so that its fluorescent signal effectively attenuated or “quenched”. This effect may be observed as the reaction progresses, by monitoring the fluorescence intensity of the sample. In particular, it is understood that as the RNA substrate is digested, the observed fluorescence intensity will decrease over time producing a profile such as the exemplary profile shown in FIG. 10C.

### Benefits and Uses

A rate-based or kinetic assay has been developed to evaluate RNase H activity. The power of the assay is underscored by the ability utilize multiple fluorophors, the application of this assay to high-throughput screening for drug development, and for rapid evaluation of kinetic constants. In combination with assays performed in a radioactive format we have shown that this assay is specific for the degradation of RNA in an RNA/DNA hybrid substrate. This assay is superior to other RNase H assays in the literature by several (gel-based and radioactive non-TCA precipitable count, and IGEN capture assay) criteria.

First, the assay is rapid and applicable to high throughput screening (HTS) in multiple well formats, including but not limited to 96-, 384- and 1536-well formats. Second, sensitivity of this assay is equal or better relative to polyacrylamide gel-based assays. This assay is orders of magnitude more sensitive than the traditional radioactivity release assay (see, e.g. Stavrianopoulos, Proc. Natl. Acad. Sci. U.S.A. 1976, 73:1087-1091; Papaphilis & Kamper, Anal. Biochem. 1985, 145:160-169; Krug & Berger, Proc. Natl. Acad. Sci. U.S.A. 1989, 86:3539-3543; Crouch et al., Methods Enzymol. 2001, 341:395413; Lima, Methods Enzymol. 2001, 341:430-440; Synder & Roth, Methods Enzymol. 2001, 341440452). Third, relative to the IGEN assay (96-well format) it is a direct determination of RNase H activity and does not rely on a capture of the product for detection of enzyme activity or inhibition of enzyme activity. Fourth, the assay is rate-based and allows for direct determination of inhibition constants. Combined, this assay provides the sensitivity of a radioactive gel-based assay with the greater speed than a radioactive release assay and does not require a second event for detection of enzyme activity as does the IGEN capture assay.

The commercial value of this assay is drug development. Modifications of this assay will allow for the development of new assays such as HIV integrase or other RNA and DNA metabolizing enzymes.

### EXAMPLES

The present invention is also described by means of the following examples. However, the use of these or other examples anywhere in the specification is illustrative only and in no way limits the scope and meaning of the invention or of any exemplified term. Likewise, the invention is not limited to any particular preferred embodiments described herein. Indeed, many modifications and variations of the invention may be apparent to those skilled in the art upon reading this specification and can be made without departing from its spirit and scope. The invention is therefore to be limited only by the terms of the appended claims along with the full scope of equivalents to which the claims are entitled

### Example 1

### Measurement of RNase H Activity in an Endpoint Assay

This example describes experiments which use an endpoint, PAGE analysis based assay to measure the activites of two exemplary RNase H enzymes: E. coli RNase H1 and HIV reverse transcriptase. In HIV, the p66/p51 reverse transcriptase (RT) holoenzyme has RNase activity which is located at the C-terminal end of the p66 subunit (Hansen et al., EMBO J. 1988, 7:239-243; Kohlstaedt et al., Science 1992, 256:1783-1790; and Sarafianos et al., EMBO J. 2001, 20:1449-1461). Mutations that effect that enzyme's RNase H activity also abolish virus infectivity (Id.), making the RNase H an attractive target for novel antiviral therapies.

Materials and Methods:

RNase H. Samples of HIV p66/p51 heterodimer were obtained from Enzyco, Inc. (Replidyne Inc., Louisville Colo.) Methods for the recombinant expression, purification and characterization of this enzyme have been previously described (Thimmig & McHenry, J. Biol. Chem. 1993, 268:16528-16536). Purity of the enzyme samples was verified on polyacrylamide gels. Its specific activity was also assayed and determined to be 27 dNTP inc/μg/60 min, which is comparable to the specific activity of other HIV RT enzymes. Samples of E. coli RNase H1 were purchased from EPICENTR (Madison, Wis.).

RNA-DNA substrate. Initial reactions used a ssRNA molecule annealed to a complementary DNA sequence. Briefly, ssRNA molecules having the nucleotide sequenced set forth in SEQ ID NO: 1 (shown below) were produced by a T7 RNA polymerase reaction using a MEGAshortscript™ High Yield Transcription Kit (Ambion Inc., Austin Tex.). Briefly, annealed oligomers (SEQ ID NOS:A and B, shown below) were used as the DNA substrate for synthesis of the RNA sequence set forth in SEQ ID NO: 1 (shown below) with a T7 RNA polymerase.

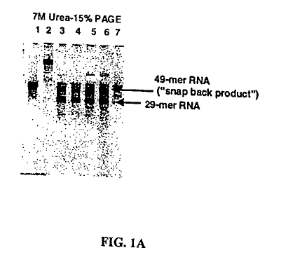

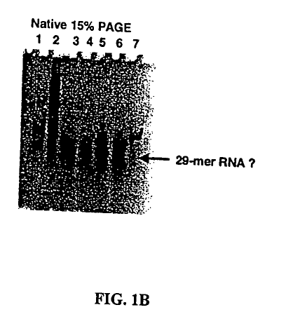

The RNA generated in this reaction was qualitatively analyzed on ethidium bromide (EtBr) stained denaturing (FIG. 1A) and non-denaturing (FIG. 1B) polyacrylamide gels. These gels resolve the desired 29mer RNA product, but also reveal significant amounts of a “snapback” RNA product estimated to be about 45 to 49 nucleotides in length.

Radiolabled RNA was generated by incorporating ³³P-ATP in the T7 RNA polymerase reaction, and annealed to an unlabeled ssDNA 49mer having the nucleotide sequence set forth in SEQ ID NO:2 (shown below). In an alternative version of these experiments, the complementary DNA oligonucleotide (SEQ ID NO:2) was radiolabeled with ³³P at the 5′ end by T4 PNK, and annealed to the unlabeled 29mer ssRNA (SEQ ID NO: 1).

| | |
|---|---|
| 5′-GACTAATACGACTCACTATAGGAAGAAAATATCATCTTTGGTGTTAACA-3′ | (SEQ ID NO:A) |
| 3′-CTGATTATGCTGAGTGATATCCTTCTTTTATAGTAGAAACCACAATTGT-5′ | (SEQ ID NO:B) |
| 5′-GGAAGAAAAUAUCAUCUUUGGUGUUAACA-3′ | (SEQ ID NO:1) |
| 5′-TGTTAACACCAAAGATGATATTTTCTTCCTATAGTGAGTCGTATTAGTC-3′ | (SEQ ID NO:2) |

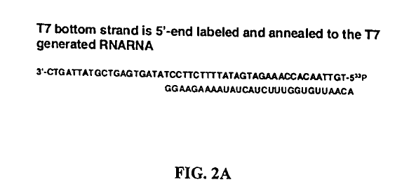

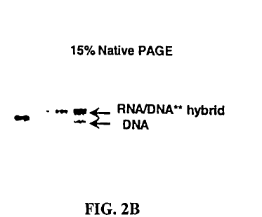

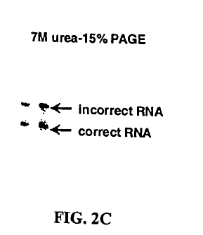

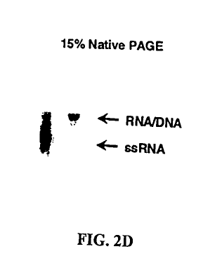

The quality of these radiolabeled RNA-DNA hybrid substrates was quantitatively evaluated on polyacrylamide gels. FIG. 2B shows the image of a non-denaturing gel loaded with the unlabeled RNA (SEQ ID NO: 1) annealed to ³³P-end labeled DNA (SEQ ID NO:2), whereas FIGS. 2C-2D show images of denaturing (FIG. 2C) and non-denaturing (FIG. 2D) gels loaded with radiolabeled RNA (SEQ ID NO: 1) annealed to unlabeled DNA (SEQ ID NO:2). Quantitative phosporimagery of the labeled RNA in denaturing gels (FIG. 2C) indicates that the contaminant “snapback” RNA represents approximately 35 to 40% of the total RNA. As expected, the “snapback” RNA species is not seen in the native (i.e., non-denaturing) gel (FIG. 2D), since separation of the RNA molecules in that gel is dependent upon both conformation and size of the different RNA species, whereas separation in the denaturing gel of FIG. 2C is independent of the native molecule's conformation.

Results:

Time dependent RNA degradation by RNase H. Aliquots containing 0.5 pmol of the radio-labeled DNA-RNA substrate (25 nM concentration) and 0.1 U of HIV reverse transcriptase (1.9 fmol at 0.095 pM concentration) were incubated in Tris buffer (pH 8) with 10 mM MgCl₂, KCl (between 0 and 30 mM), 3% glycerol, 0.2% NP-40, 50 μg/ml BSA and 1 mM DTT. In a parallel experiment, aliquots containing 0.5 pmol of the labeled DNA-RNA substrate (25 nM concetration) were also incubated with 0.01 U of E. coli RNase H1 enzyme in Tris buffer (pH 7.5), containing 100 mM NaCl, 10 mM MgCl₂, 3% glycerol, 0.02% NP-40 and 50 μg/ml BSA. The various aliquots were incubated at 37° C. for 0 (i.e., <30 seconds), 5, 10, 20, 30 40 and 60 minutes to allow for RNA degradation by the RNase H enzymes, after which time the reactions were quenched by the addition of an equal volume of 100 mM EDTA. The reaction products were analyzed by PAGE.

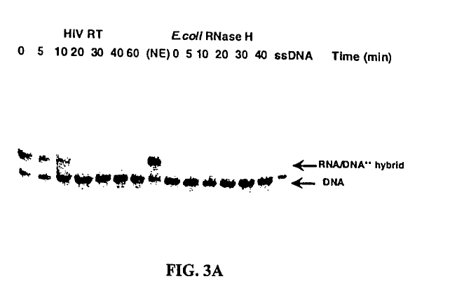

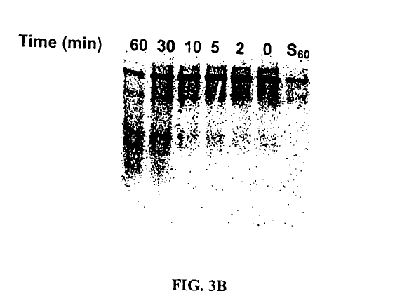

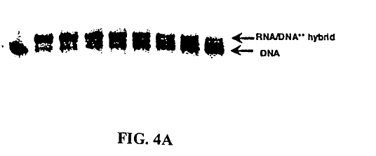

The results are presented in FIGS. 3A-3B. In particular, FIG. 3A shows the image of a polyacrylamide gel run for a substrate of unlabeled RNA/end-labeled DNA digested with HIV RT RNase H (lanes 1-7) and E. coli RNase H1 (lanes 9-14). Lane 8 shows results from a control experiment where no enzyme was present (NE). As expected, the intensity of bands corresponding to the RNA-DNA hybrid decreases as aliquots are incubated for longer times, while the intensity of bands corresponding to labeled DNA alone increases.

FIG. 3B shows the image of an identical polyacrylamide gel run for a labeled RNA/unlabeled DNA hybrid substrate digested with the 3U HIV RT RNase (66 fmol in 6.6 nM) with 50 mM HEPES (pH 8), 10 mM MgOAc, 0.02% NP40, 5 μg/μl BSA, 3% glycerol and 2 mM DTT. The cleavage products of the RNase enzyme are visible and are degraded in a similar rate dependent manner as in FIG. 3A.

To investigate the assay's ability to distinguish different levels of RNase activity, additional experiments were performed using different concentrations of the HIV-RT enzyme and/or substrate. 150 nM of unlabeled RNA/end-labeled DNA hybrid substrate was incubated with either 0.3 or 0.1 U of HIV-RT enzyme under the conditions described, supra.

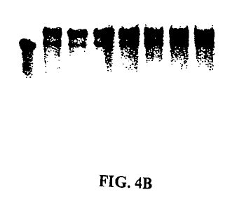

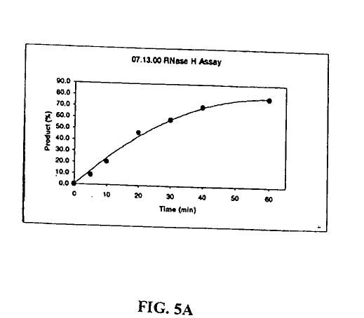

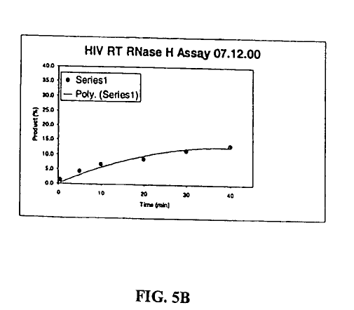

Quantitative results from those experiments are shown in FIGS. 4A-B. In particular, FIG. 4A shows the image of a polyacrylamide gel loaded with substrate that was digested with 0.3 U of HIV-RT enzyme, whereas FIG. 4B shows the image of a polyacrylamide gele loaded with substrate digested by 0.1 U of the HIV-RT enzyme. Quantitative plots of these data, showing the % of ssDNA observed as digestion progressed with time, are provided in FIGS. 5A-B, respectively. As expected, the assay detected lower levels of digestion over identical periods of time when lower RNase enzyme concentration was used.

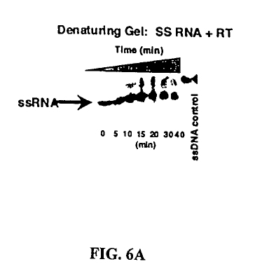

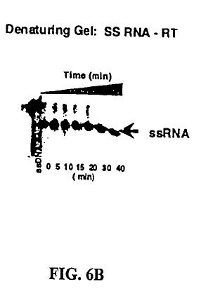

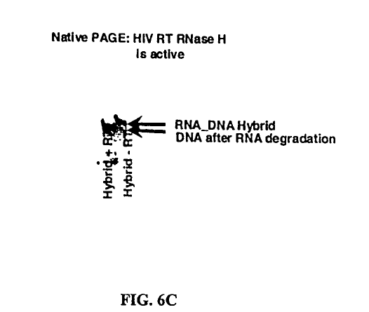

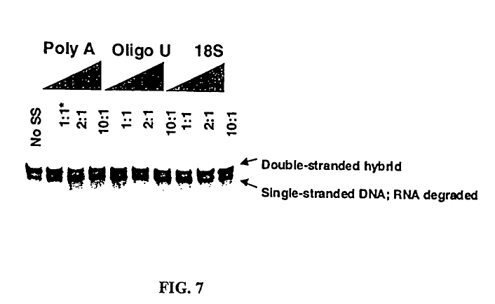

HIV RT RNase H does not degrade ssRNA. Experiments were also performed to determine whether there may be any non-specific RNA degradation by the RNase H enzyme which might have effected the above discussed results. Here, 0.1 μM aliquots of radiolabeled ssRNA substrate were incubated with 1 U HIV RT RNase H enzyme under the conditions described for the previous experiments, supra, and the reaction products were run on denaturing polyacrylamide gels (FIG. 6A). As a control, identical ssRNA aliquots were incubated under the same condition but without RNase H, and these control aliquots were also run on denaturing gels (FIG. 6B). The amount of radiolabled RNA substrate detected is similar for each reaction time and smaller degradation products are not observed, indicating that ssRNA is not degraded by the RNase H enzyme. The extent of RNase H activity was monitored in a parallel experiment with an RNA-DNA hybrid substrate (FIG. 7C) and confirms that the enzyme used in these experiments was functional.

Single stranded DNA and RNA contaminants do not affect RNase H activity. Experiments were also performed to determine whether ssRNA and/or ssDNA contaminants or reaction products might affect measurements of RNase H activity, e.g., by inhibiting that enzyme. First, aliquots containing 0.1 nM (5 pmol) of the RNA-DNA hybrid substrate and 1 U (2.2 ng or 3 fmol) of the HIV RT enzyme were incubated with 5, 10 and 50 pmol of either homopolymeric polyA (SEQ ID NO:3) or polyU (SEQ ID NO:4) or heteropolymeric (18S) RNA (SEQ ID NO:5), so that the molar ratios to RNA-DNA hybrid substrate were 1:1, 2:1 and 10:1, respectively.

| | | |
|---|---|---|
| homopolymeric polyA | 5′-(A)ₙ-3′ | (n ≈ 500 to 1000) |
| (SEQ ID NO:3) | | |
| homopolymeric polyU | 5′-(U)ₙ-3′ | (n ≈ 500 to 1000) |
| (SEQ ID NO:4) | | |

| | |
|---|---|
| 18S RNA | 5′-CCCUCUCUCUCUCUUAAUGGGAGUGAUUUCCCUCCUCUU |
| (SEQ ID NO:5) | CGAAUAGGGUUCUAGGUUGAUGCUCGAAAAAUUGACGUCG |
| | UUGAAAUUAUAUGCGAUAACCUCGACCUUAAAGGCGCCGAC |
| | GACAAG-3′ |

Each aliquot was incubated at 37° C. and the reaction products were run on polyacrylamide gels (FIG. 7). Titration of the sample with 125-mer 18S RNA, which contains significant secondary structure, did inhibit HIV RT RNase H in a dose dependent manner, as determined by the measured amount of end-labeled ssDNA after each reaction. However, such contaminants are unlikely to be present in any “real” RNase H assay. The homopolymeric U and A, which do not exhibit any secondary structure, did not inhibit HIV RT RNase H activity.

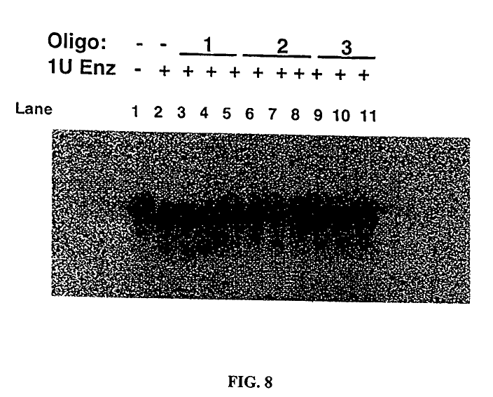

Similar experiments were also performed aliquots containing 0.1 μM (5 pmol) of the RNA-DNA hybrid substrate and 1 U (2.2 ng or 19 fmol) of HIV RT enzyme were incubated with one of single stranded DNA oligonucleotides set forth in Table I, below. These oligonucleotides, which are referred to here as Oligo 1, Oligo 2 and Oligo 3 are also identified by SEQ ID NOS:6-8, respecitvely. The molar ratio of each ssDNA oligomer to substrate in the different aliquots was 1:1, 2:1 and 10:1 (i.e., 5, 10 and 50 pmol). Again, the aliquots were incubated at 37° C. to permit RNA degradation by the RNase H, and then quenched after 30 minutes and analyzed by PAGE (FIG. 8) The results indicate the HIV RT RNase H activity is not inhibited by ssDNA. Thus, the presence of single-stranded RNA or ssDNA in the assay (generated, e.g., as a consequences of enzyme activity) will only minimally effect the assessment of RNase activity, if at all.

TABLE I

Deoxyoligonucleotide Sequences Titrated
with RNase H Substrate

| | | |
|---|---|---|
| Oligo 1 | (SEQ ID NO:6) | 5′-GTGAGGGTAATTCTCTCTCTCTCCCAAACCCCAAA-3′ |
| Oligo 2 | (SEQ ID NO:7) | 5′-ATCTTGGGATAAGCTTCTCCTCCC-3′ |
| Oligo 3 | (SEQ ID NO:8) | 5′-TTGCTGCAGTTAAAAAGCTCGTAG-3′ |

RNA degradation requires competent RNase H activity. To confirm that the RNA degradation observed in these experiments is actually due to RNase H and not some other activity of the RT holoenzyme, assays were performed using RT enzyme from different sources. Specifically 0.1 μM (5 pmol) of the RNA-DNA substrate was incubated with either 1 U of the HIV RT enzyme (2.2 ng or 19 fmol), 1 U of MMLV RT enzyme (10 ng or 15 fmol) obtained from Promega (Madison, Wis.). An identical experiment was also performed using an equivalent amount of a mutant MMLV RT enzyme that has been previously described and characterized as having no RNase H activity (Roth et al., J. Biol. Chem. 1985, 260:9326; Tanese et al., Proc. Natl. Acad. Sci. U.S.A. 1988, 85:1977).

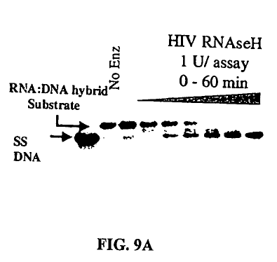

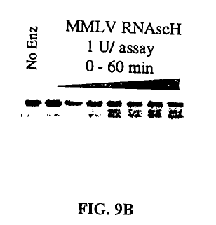

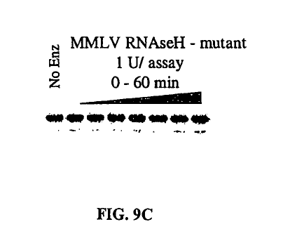

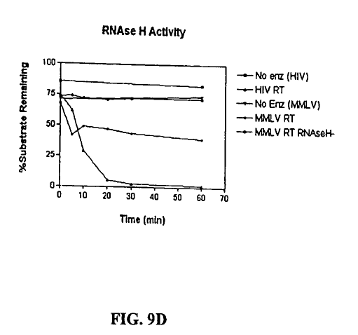

Aliquots of each sample were incubated at 37° C. for <30 seconds, 10, 20, 30 and 60 minutes, after which time the reaction was quenched and reaction products were analyzed by PAGE as described, supra, in the previous experiments. The results from the experiments are shown in FIG. 9A (HIV RNase H), FIG. 9B (MMLV RNase H) and FIG. 9C (MMLV RNase H-mutant). The amount of substrate remaining in each aliquot after the reaction was quantitatively determined by volume analysis following the phosphorimagry, using the formula:

% substrate remaining=((substrate)/(substrate+product))×100%

The results from this quantitative analysis are plotted in FIG. 9D, and confirm that the apparent degradation of RNA from RNA-DNA hybrids observed in these assays is the result of a functional RNase H activity.

### Example 2

### Real Time Assay for RNase H Activity

This example demonstrates particular embodiments of a preferred assay that is capable of detecting and monitoring RNase H activity in real time. The exemplary assay uses an RNA-DNA hybrid substrate that comprises a fluorophor moiety and a quencher moiety. The fluorophor moiety comprises a moiety that is capable of emitting a fluorescent or other detectable signal. The quencher moiety, by contrast, comprises a moiety that is capable of absorbing the signal generated by the fluorophor moiety.

For instance, in the exemplary embodiment described here the fluorophor moiety is fluorescein and the quencher moiety is dabcyl, both of which are commercially available, e.g., from Stratagene (La Jolla, Calif.). However, the precise identity of the fluorescent and quencher moieties is not critical and a variety of such moieties which can be used for the present invention are commercially available and/or generally known in the art. Examples of other common fluorophors that can be used include but are not limited to Cy3, Cy3.5, Cy5 and Cy5.5 (available from Amersham Biosciences Corp., Piscataway N.J.) as well as Texas red, fluoroscein, 6-FAM, HEX, TET, TAMRA, Rhodamine Red, Rhodamine Green, Carboxyrhodamine, BODIPY, 6-SOE, Coumarin and Oregon Green, all of which are commercially available, e.g., from Molecular Probes (Eurgene, Oreg.) or Sigma-Aldrich Corp. (St. Louis, Mo.). Exemplary quencher moieties include DABCYL (available from Sigma-Aldrich Corp., St. Louis Mo. or from Molecular Probes, Eugene Oreg.) as well as Black Hole Quenchers (“BSQs”, available from Biosearch Technologies, Inc., Novato Calif.) such as BHQ-1, BHQ-2 and BHQ-3.

An exemplary embodiment of a DNA-RNA hybrid substrate which may be used in such an assay is schematically illustrated in FIG. 10A. In this example, the DNA substrate comprises the nucleotide sequence set forth in SEQ ID NO:9, whereas the RNA substrate comprises the nucleotide sequence set forth in SEQ ID NO: 10. Those skilled in the art will appreciate that the exact sequence of the DNA-RNA substrate is not critical for practicing the invention. However, the sequences will preferably have certain properties. In particular, the sequence of the DNA substrate preferably comprises a 5′-region and a 3′-region, which are located at deoxyoligonucleotide's 5′- and 3′-ends, respectively. Preferably, the 5′-region and 3′-region are complementary and capable of hybridizing to each other under assay conditions. The DNA substrate also preferably comprises a center region that is complementary to at least a part of the RNA substrate so that the DNA substrate and RNA substrate are capable of hybridizing to each other under assay conditions, thereby forming the DNA-RNA hybrid substrate. For illustrative purposes, the exemplary DNA substrate is illustrated in FIG. 10A as having the fluorophor moiety attached to the 3′-region (e.g., on the 3′-end of the deoxyoligonucleotide) and having the quencher moiety attached to the 5′-region (e.g., on the 5′-end of the deoxyoligonucleotide). However, embodiments in which the quencher moiety is attached to the 3′-region (e.g., on the 3′-end of the deoxyoligonucleotide) and the fluorophor moiety is attached to the 5′-region (e.g., on the 5′-end of the deoxyoligonucleotide) are also contemplated and generally will be equally preferred.

Without being limited to any particular theory or mechanism of action, it is believed that as RNase H degrades RNA in the RNA-DNA hybrid substrate, the 5′- and 3′-regions of the DNA anneal to each other so that the oligonucleotide probe adopts a conformation such as that illustrated in FIG. 10B, placing the fluorophor moiety and the quencher moiety in sufficient proximity so that the quencher moiety absorbs at least part of the detectable signal emitted by the fluorophor moiety. Consequently, RNase H activity may be detected and monitored by detecting an attenuation or decrease in fluorescence (FIG. 10C).

To demonstrate its efficacy, both HIV RT RNase H and E. coli RNase H1 were examined using this assay format. RNase H enzymes and RNA substrate (SEQ ID NO: 10) were prepared as described in Example 1, above. A DNA oligonucleotide probe (SEQ ID NO:9) was also prepared according to routine methods and labeled on the 3′-end with fluorecein and with dabcyl on the 5′-end, both of which are available from Stratagene (La Jolla, Calif.).

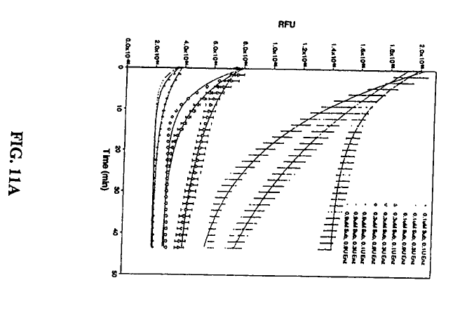

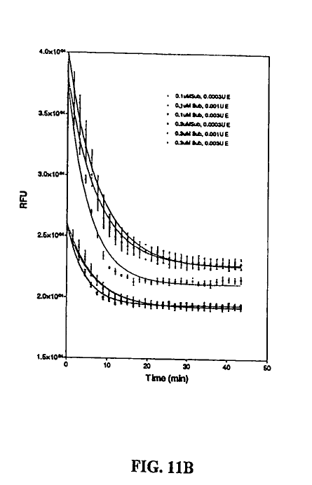

In a first set of experiments, an oligonucleotide probe (SEQ ID NO:9) labeled with the fluorophor Texas red and a DABCYL quencher moiety was annealed to RNA (SEQ ID NO: 10) at molar ratios of 1:1 and 1:2 (DNA:RNA). Each assay was carried out at 25° C. in a final volume of 25 μl of 50 mM Tris buffer (pH 8) with 10 mM MgCl₂, optional KCl (0 to 30 mM), 3% glycerol, 1 mM DTT, 0.02% NP-40 and 50 μg/ml BSA containing substrate and inhibitor at the indicated quantities or concentrations. Substrate hydrolysis was monitored during the reaction as a function of time using a Wallac Victor fluorescence microplate reader (Perkin Elmer Life Sciences, Inc., Boston Mass.) with excitation and emission wavelengths set with filters at 585 and 615 nm, respectively, and with a 10 nm band pass. The substrate was added to the enzyme sample to initiate the reaction. Instrument data collection was monitored with a personal computer compatible with 32-bit Windows Workstation software, designed to utilize the full capabilities of Windows™ 95/98/NT. Fluorescent measurements were taken every 30 seconds and are plotted in FIG. 11A. A similar set of experiments were also performed in which 0.1 μM and 0.3 μM of the DNA-RNA substrate were incubated with 0.0003 and 0.001 U of E. coli RNase H1 in 50 mM Tris buffer (pH 7.5) containing 100 mM NaCl, 10 mM MgCl2, 3% glycerol, 0.02% NP40, 50 μg/ml BSA. The fluorescent signal measured in these samples is plotted as a function of real time in FIG. 11B.

These data show that the above-described assay format is robust and effective. A decrease in the fluorescence signal is observed that is a function of both the incubation time and enzyme concentration, and is consistent with the rate of RNA degradation by the enzyme.

### REFERENCES CITED

Numerous references, including patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entirety and to the same extent as if each reference was individually incorporated by reference.

What is claimed is:

1. A method for detecting a nuclease-mediated cleavage of a target nucleic acid, which method comprises:
(a) hybridizing a target nucleic acid to a fluorescently labeled oligonucleotide probe complementary to the target nucleic acid and containing a flourophor at one terminus and a quenching group at the other terminus, wherein (i) when the probe is unhybridized to the target nucleic acid, the probe adopts a conformation that places the flourophor and quencher in such proximity that the quencher quenches the flourescent signal of the flourophor and (ii) formation of the probe-target hybrid causes sufficient separation of the flourophor and quencher to reduce quenching of the flourescent signal of the flourophor;
(b) contacting the probe-target hybrid with an agent having nuclease activity in an amount sufficient to selectively cleave the target nucleic acid and thereby release the intact probe; and
(c) detecting the release of the probe by measuring a decrease in the flourescent signal of the flourophor as compared to the signal of the probe-target hybrid.

2. The method of claim 1, wherein the agent is an enzyme having an RNase H activity.

3. The method of claim 2, wherein the agent is selected from the group consisting of HIV reverse transcriptase, E. coli RNase H1, E. coli RNase H2, Human RNase H1, Human RNase H2, hammerhead ribozyme, HBV reverse transcriptase, and integrase.

4. The method of claim 1, wherein the probe is DNA, and the target is the DNA:RNA hybrid substrate.

5. The method of claim 1, wherein the probe is at least 18 nucleotides in length.

6. The method of claim 1, wherein the probe, when unhybridized to the target nucleic acid, adopts a hairpin secondary structure conformation that brings the fluorophor and quencher into proximity.

7. The method of claim 1, wherein the nuclease reaction is performed in the presence of a compound, wherein a difference in the rate of the decrease in the flourescent signal of the flourophor during the nuclease reaction, as compared to the decrease observed when the same reaction is conducted in the absence of the compound, is indicative of the ability of the compound to either inhibit or enhance the nuclease activity of the agent.

8. The method of claim 1, which further comprises monitoring the flourescent signal of the flourophor during the nuclease reaction.

9. A method for measuring a RNase H activity of an agent, which method comprises:
(a) hybridizing a target RNA to a fluorescently labeled oligodesoxyribonucleotide probe complementary to the target RNA and containing a flourophor at one terminus and a quenching at the other terminus, wherein (i) when the probe is unhybridized to the target RNA, the probe adopts a conformation that places the flourophor and quencher in such proximity that the quencher quenches the flourescent signal of the flourophor and (ii) formation of the probe-target hybrid causes sufficient separation of the flourophor and quencher to reduce quenching of the flourescent signal of the flourophor;
(b) contacting the probe-target hybrid with the agent in an amount sufficient to selectively cleave the target RNA and thereby release the intact probe; and
(c) measuring a decrease in the flourescent signal of the flourophor as compared to the signal of the probe-target hybrid.

10. The method of claim 9, wherein the agent is an enzyme having an RNase H activity.

11. The method of claim 10, wherein the agent is selected from the group consisting of HIV reverse transcriptase, E. coli RNase H1, E. coli RNase H2, Human RNase H1, Human RNase H2, hammerhead ribozyme, HBV reverse transcriptase, and integrase.

12. The method of claim 9, wherein the probe is at least 18 nucleotides in length.

13. The method of claim 9, wherein the probe, when unhybridized to the target RNA, adopts a hairpin secondary structure conformation that brings the fluorophor and quencher into proximity.

14. The method of claim 9, wherein the RNase H-mediated reaction is performed in the presence of a compound, wherein a difference in the rate of the decrease in the flourescent signal of the flourophor during the RNase H-mediated reaction, as compared to the decrease observed when the same reaction is conducted in the absence of the compound, is indicative of the ability of the compound to either inhibit or enhance the RNase H activity of the agent.

15. The method of claim 9, which further comprises monitoring the flourescent signal of the flourophor during the RNase H-mediated reaction.

16. A method of screening for a modulator of the nuclease activity of an agent, which method comprises:
(a) hybridizing a target nucleic acid to a fluorescently labeled oligonucleotide probe complementary to the target nucleic acid and containing a flourophor at one terminus and a quenching group at the other terminus, wherein (i) when the probe is unhybridized to the target nucleic acid, the probe adopts a conformation that places the flourophor and quencher in such proximity that the quencher quenches the flourescent signal of the flourophor and (ii) formation of the probe-target hybrid causes sufficient separation of the flourophor and quencher to reduce quenching of the flourescent signal of the flourophor;
(b) preparing two samples containing the probe-target hybrid;
(c) contacting the probe-target hybrid of a first sample with the agent in an amount sufficient to selectively cleave the target nucleic acid and thereby release the intact probe;
(d) contacting the probe-target hybrid of a second sample with the agent in an amount sufficient to selectively cleave the target nucleic acid and thereby release the intact probe in the presence of a candidate compound, which is being tested for its ability to modulate the nuclease activity of the agent;
(e) detecting the release of the probe in each sample by measuring a decrease in the flourescent signal of the flourophor as compared to the signal of the probe-target hybrid; and
(f) comparing the rate of the decrease in the flourescent signal of the flourophor in the two samples, wherein a difference in the rate of the decrease in the flourescent signal of the flourophor during the nuclease reaction in the two samples is indicative of the ability of the compound to either inhibit or enhance the nuclease activity of the agent.

17. The method of claim 16, wherein a greater extent or relative rate of decrease of the flourescent signal of the flourophor in the second sample compared to the first sample indicates that the candidate compound is an agent agonist.

18. The method of claim 16, wherein a lesser extent or relative rate of decrease of the flourescent signal of the flourophor in the second sample compared to the first sample indicates that the candidate compound is an agent antagonist.

19. A kit for measuring a nuclease activity of an agent, comprising a target nucleic acid and a fluorescently labeled oligonucleotide probe complementary to the target nucleic acid and containing a flourophor at one terminus and a quencher at the other terminus, wherein (i) when the probe is unhybridized to the target nucleic acid, the probe adopts a conformation that places the flourophor and quencher in such proximity that the quencher quenches the flourescent signal of the flourophor and (ii) formation of the probe-target hybrid causes sufficient separation of the flourophor and quencher to reduce quenching of the flourescent signal of the flourophor.

20. The kit of claim 19, wherein the probe is at least 18 nucleotides in length.

21. The kit of claim 19, wherein the probe, when unhybridized to the target nucleic acid, adopts a hairpin secondary structure conformation that brings the fluorophor and quencher into proximity.

22. The kit of claim 19, wherein the probe is DNA, and the target nucleic acid is DNA:RNA hybrid substrate.

23. The kit of claim 19, further comprising the agent.

24. The kit of claim 23, wherein the agent is is selected from the group consisting of RNase H, reverse transcriptase, E. coli RNase H1 and H2, Human RNase H1 and H2, hammerhead ribozymes, HBV reverse transcriptase, and integrase.

25. The kit of claim 23, wherein the reverse transcriptase is HIV reverse transcriptase.

26. An assay mixture for measuring a nuclease activity of an agent, comprising a target nucleic acid and a fluorescently labeled oligonucleotide probe complementary to the target nucleic acid and containing a flourophor at one terminus and a quenching group at the other terminus, wherein (i) when the probe is unhybridized to the target nucleic acid, the probe adopts a conformation that places the flourophor and quencher in such proximity that the quencher quenches the flourescent signal of the flourophor and (ii) formation of the probe-target hybrid causes sufficient separation of the flourophor and quencher to reduce quenching of the flourescent signal of the flourophor.

27. The assay mixture of claim 26, wherein the probe is DNA, and the target nucleic acid is RNA.

28. The assay mixture of claim 26, wherein the probe and the target nucleic acid are hybridized to each other to form a probe-target hybrid.

29. The assay mixture of claim 28, further comprising the agent.

30. The assay mixture of claim 29, wherein the agent is selected from the group consisting of RNase H, reverse transcriptase, E. coli RNase H1 and H2, Human RNase H1 and H2, hammerhead ribozymes, HBV reverse transcriptase, and integrase.

31. The assay mixture of claim 30, wherein the reverse transcriptase is HIV reverse transcriptase.


SEQUENCE LISTING (8 sequences)

SEQ ID NO: 1 - RNA, 29 nt, Artificial Sequence
  synthetic RNA
ggaagaaaauaucaucuuugguguuaaca

SEQ ID NO: 2 - DNA, 49 bp, Artificial Sequence
  synthetic DNA
tgttaacaccaaagatgatattttcttcctatagtgagtcgtattagtc

SEQ ID NO: 3 - RNA, 126 nt, Artificial Sequence
  synthetic RNA
cccucucucucucuuaaugggagugauuucccuccucuucgaauaggguucuagguugaugcucgaaaaauugacgucguugaaauuauaugcgauaaccucgaccuuaaaggcgccgacgacaag

SEQ ID NO: 4 - DNA, 35 bp, Artificial Sequence
  oligonucleotide
gtgagggtaattctctctctctcccaaaccccaaa

SEQ ID NO: 5 - DNA, 24 bp, Artificial Sequence
  oligonucleotide
atcttgggataagcttctcctccc

SEQ ID NO: 6 - DNA, 24 bp, Artificial Sequence
  oligonucleotide
ttgctgcagttaaaaagctcgtag

SEQ ID NO: 7 - DNA, 33 bp, Artificial Sequence
  oligonucleotide
acgagcaacaccaaagatgatattttcgctcgc

SEQ ID NO: 8 - DNA, 49 bp, Artificial Sequence
  oligomer
gactaatacgactcactataggaagaaaatatcatctttggtgttaaca